(19) United States
(12) Patent Application Publication
(10) Pub. No.: US 2024/0268839 A1
(43) Pub. Date: Aug. 15, 2024

(54) PROSTHETIC IMPLANT REVISION TOOLS

(71) Applicant: Simplex Designs, LLC, Duluth, GA (US)

(72) Inventor: Jose S. RIVERA, JR., Naples, FL (US)

(73) Assignee: Simplex Designs, LLC, Duluth, GA (US)

(21) Appl. No.: 18/434,876

(22) Filed: Feb. 7, 2024

Related U.S. Application Data
(60) Provisional application No. 63/444,710, filed on Feb. 10, 2023.

(51) Int. Cl. A61B 17/16

(52) U.S. Cl. CPC A61B 17/1675; A61B 17/1631; A61B 2217/002

(57) ABSTRACT

A tool for removal of a tibial implant a proximal end, and a distal end having an elongated cutting portion, wherein the elongated cutting portion has an edge sharpened for cutting extending a predetermined distance on a first side of the tool, around the distal end, and extending the predetermined distance on a second side of the tool. The tool is configured to release an implanted prosthesis by closely following the bone/implant interface. For example, the tools are used to cut around, dislodge, and remove a tibial component of a prosthetic knee. The edge is situated to be able to cut in many directions while inserted in the knee.

### CROSS-REFERENCE TO RELATED APPLICATION

This application claims the benefit of U.S. Provisional Application No. 63/444,710, filed Feb. 10, 2023, which is incorporated herein by reference in its entirety.

### FIELD OF THE DISCLOSURE

The present disclosure relates to an apparatus and associated methodology for prosthetic implant revision tools.

### BACKGROUND

Joint arthroplasty is increasingly common in the United States and around the world. Arthroplasty can involve the complete or partial replacement of hips, knees, or shoulders. During primary total knee replacement, the knee joint is replaced with an implant, or prosthesis, made of metal and plastic components. Although most total knee replacements are very successful, over time, problems such as implant wear and loosening may require a revision procedure to replace the original components. For example, in order for a total knee replacement to function properly, an implant must remain firmly attached to the bone. During the initial surgery, the implant was either cemented into the bone or press-fit to allow bone to grow onto the surface of the implant. In either case, the implant was firmly fixed. Over time, however, an implant may loosen from the underlying bone, causing the knee to become painful.

During revision surgeries surgeons attempt to remove the existing implants while minimizing damage to surrounding bone and tissue. This is often a difficult task as implants are designed to join with the surrounding bone over time Minimizing the loss of this bone during a revision helps the new implant to be properly affixed. It also reduces the length and cost of the revision surgery and further reduces recovery time. Efforts have been made over the years to provide tools that aid in the efficient removal of a prosthetic. However, the existing devices and techniques all suffer from significant drawbacks. Namely, existing devices rely heavily upon the technique of the surgeon and do not include tools that adequately accommodate the shape of the prosthetic being removed or that otherwise minimize bone loss. The implant revision tools of the present disclosure are aimed at overcoming these and other shortcomings present in the background art.

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

### SUMMARY

According to aspects of the disclosed subject matter, a tool for removal of a tibial implant a proximal end, and a distal end having an elongated cutting portion, wherein the elongated cutting portion has an edge sharpened for cutting extending a predetermined distance on a first side of the tool, around the distal end, and extending the predetermined distance on a second side of the tool. The tool is configured to release an implanted prosthesis by closely following the bone/implant interface. For example, the tools are used to cut around, dislodge, and remove a tibial component of a prosthetic knee. The edge is situated to be able to cut in many directions while inserted in the knee.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 illustrates a perspective view of a straight osteotome according to one or more aspects of the disclosed subject matter;

FIG. 2 illustrates the straight osteotome inserted under a tibial implant according to one or more aspects of the disclosed subject matter;

FIG. 3 illustrates a perspective view of curved lateral osteotomes according to one or more aspects of the disclosed subject matter;

FIG. 4 illustrates a perspective view of the curved lateral osteotomes according to one or more aspects of the disclosed subject matter;

FIG. 5 illustrates exemplary curved lateral osteotomes inserted under a tibial implant according to one or more aspects of the disclosed subject matter;

FIG. 6 illustrates a perspective view of a posterior osteotome with a radial blade according to one or more aspects of the disclosed subject matter;

FIG. 7 illustrates the posterior osteotome with the radial blade inserted under a tibial implant according to one or more aspects of the disclosed subject matter;

FIG. 8 illustrates a perspective view of an offset osteotome according to one or more aspects of the disclosed subject matter;

FIG. 9 illustrates the offset osteotome inserted under a tibial implant according to one or more aspects of the disclosed subject matter;

FIG. 10 illustrates a perspective view of a tibial extractor according to one or more aspects of the disclosed subject matter;

FIG. 11 illustrates a perspective view of the tibial extractor from below according to one or more aspects of the disclosed subject matter;

FIG. 12 illustrates a perspective view of a femoral and tibial extractor according to one or more aspects of the disclosed subject matter; and

FIG. 13 illustrates a perspective view of the femoral and tibial extractor according to one or more aspects of the disclosed subject matter.

### DETAILED DESCRIPTION

The description set forth below in connection with the appended drawings is intended as a description of various embodiments of the disclosed subject matter and is not necessarily intended to represent the only embodiment(s). In certain instances, the description includes specific details for the purpose of providing an understanding of the disclosed subject matter. However, it will be apparent to those skilled in the art that embodiments may be practiced without these specific details. In some instances, well-known structures and components may be shown in block diagram form in order to avoid obscuring the concepts of the disclosed subject matter.

Reference throughout the specification to “one embodiment” or “an embodiment” means that a particular feature, structure, characteristic, operation, or function described in connection with an embodiment is included in at least one embodiment of the disclosed subject matter. Thus, any appearance of the phrases “in one embodiment” or “in an embodiment” in the specification is not necessarily referring to the same embodiment. Further, the particular features, structures, characteristics, operations, or functions may be combined in any suitable manner in one or more embodiments. Further, it is intended that embodiments of the disclosed subject matter can and do cover modifications and variations of the described embodiments.

It must be noted that, as used in the specification and the appended claims, the singular forms “a,” “an,” and “the” include plural referents unless the context clearly dictates otherwise. That is, unless clearly specified otherwise, as used herein the words “a” and “an” and the like carry the meaning of “one or more.” Additionally, it is to be understood that terms such as “left,” “right,” “top,” “bottom,” “front,” “rear,” “side,” “height,” “length,” “width,” “upper,” “lower,” “interior,” “exterior,” “inner,” “outer,” and the like that may be used herein, merely describe points of reference and do not necessarily limit embodiments of the disclosed subject matter to any particular orientation or configuration. Furthermore, terms such as “first,” “second,” “third,” etc., merely identify one of a number of portions, components, points of reference, operations and/or functions as described herein, and likewise do not necessarily limit embodiments of the disclosed subject matter to any particular configuration or orientation.

Referring now to the drawings, wherein like reference numerals designate identical or corresponding parts throughout the several views:

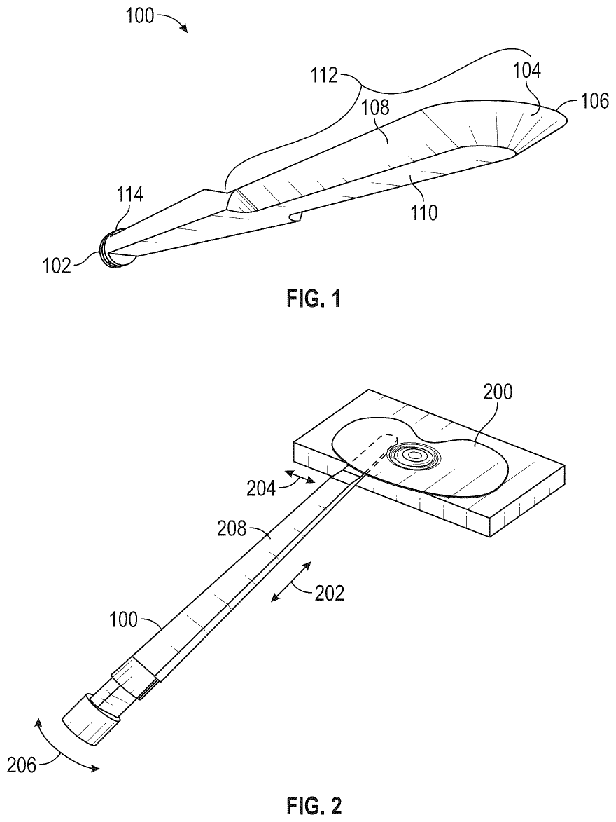

Generally speaking, the tools in FIGS. 1-13 are specifically configured to release an implanted prosthesis by closely following the bone/implant interface. For example, the tools are used to cut around, dislodge, and remove a tibial component of a prosthetic knee. The tibial component typically includes a tray and a stem, where the stem is inserted in a shin bone and the femur sits in the tray. In one or more aspects, a spacer can be included between the femur and the tray.

FIG. 1 illustrates a perspective view of a straight osteotome 100 according to one or more aspects of the disclosed subject matter; and FIG. 2 illustrates the straight osteotome 100 inserted under a tibial implant 200 according to one or more aspects of the disclosed subject matter. It should be appreciated that the reference herein related to the knee revision and the tibial implant 200 would also apply if the tibial implant were in a patient's knee. It is simply being shown outside the knee for ease of illustration and corresponding discussion. With reference to FIG. 1 and FIG. 2, the straight osteotome 100 includes proximal and distal ends (102 and 104), with distal end 104 having an edge 106 that is inserted into a knee for prosthetic implant revision. The edge 106 is sharpened for cutting. For example, the edge 106 is a radial blade. The edge 106 extends a predetermined distance along a first side of the osteotome 100, around the distal end 104, and extends the predetermined distance along a second side of the osteotome 100. In other words, the edge 106 defines an elongated cutting portion 112 of the osteotome 100. The sharpened edge 106 cuts the bone growth along the bone/implant interface and otherwise allows for the insertion of the straight osteotome 100 into the knee. In one aspect, the straight osteotome 100 is used for any area of the tibial implant other than on a posterior side.

The elongated cutting portion 112 has a flat top 108 that tapers to a flat bottom 110. As a result, the edge 106 is ideally situated to be able to cut in many directions while inserted in the knee. For example, considering movement that is parallel to the flat bottom 110, for example, the edge 106 allows the elongated cutting portion 112 to cut in a forward-backward direction corresponding to arrow 202, a left-right direction corresponding to arrow 204, and/or a rotational direction corresponding to arrow 206 while inserted in the knee.

The proximal end 102 of the osteotome 100 includes a connection portion 114 that can be connected to other tools (e.g., an impact hammer, a slap hammer, etc.). In one aspect, the connection portion 114 can be the implant removal tool connection interface described in Provisional Application No. 63/411,417 filed on Sep. 29, 2022, which is incorporated herein by reference in its entirety.

Between the elongated cutting portion 112 at the distal end and the connection portion 114 at the proximal end 104 is a shaft 208. Additionally, a height of the elongated cutting portion 112 is shorter than a height of the shaft 208.

It should be appreciated that the straight osteotome 100 can also be used for femoral knee implant removal, and it is advantageous for the same reasons described herein for tibial implant removal.

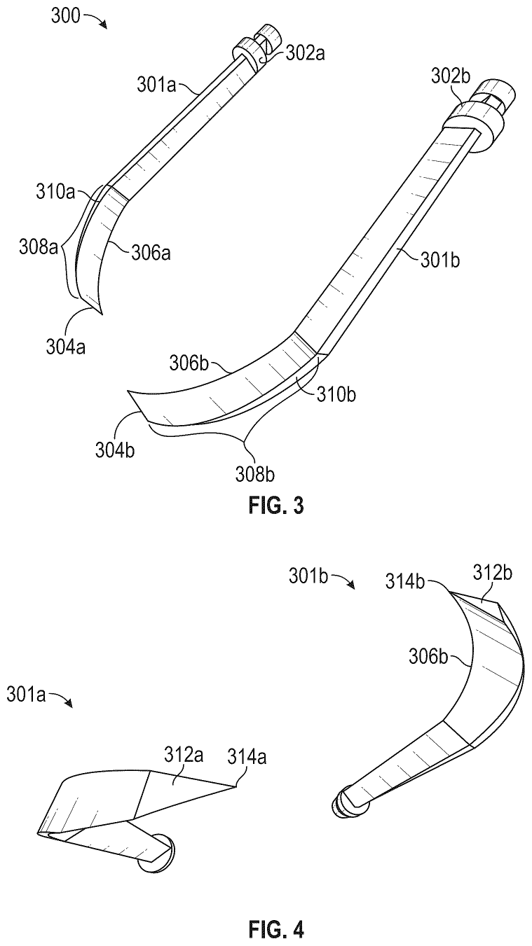

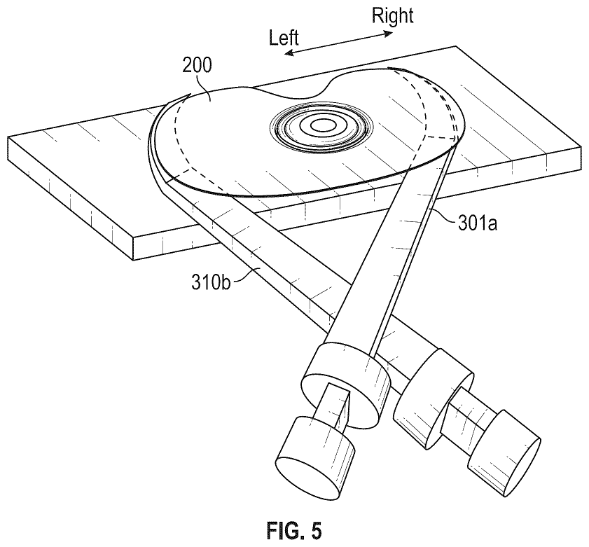

FIG. 3 illustrates a perspective view of curved lateral osteotomes 300 according to one or more aspects of the disclosed subject matter; FIG. 4 illustrates a perspective view of the curved lateral osteotomes 300 according to one or more aspects of the disclosed subject matter; and FIG. 5 illustrates exemplary curved lateral osteotomes 300 inserted under the tibial implant 200 according to one or more aspects of the disclosed subject matter. With reference to FIGS. 3, 4, and 5, the curved lateral osteotomes 300 includes a first lateral osteotome 301a and a second later osteotome 301b that can be used to cut under the tibial tray. In one aspect, the curved lateral osteotomes 300 are used to cut under the lateral sides and a posterior side of the tibial implant. The first lateral osteotome 301a includes proximal and distal ends (302a and 304a), with distal end 304a being inserted into a knee for prosthetic implant revision Similarly, the second lateral osteotome 301b includes proximal and distal ends (302b and 304b), with distal end 304b being inserted into a knee for prosthetic implant revision. The distal ends 304a,b of the curved lateral osteotomes 301a,b include an elongated cutting portion 308a,b that is curved a predetermined amount. A first side of the elongated cutting portion 308a,b is an edge 306a and 306b, respectively, that is sharpened for cutting. The sharpened edge 306a,b cuts the bone growth along the bone/implant interface and otherwise allows for the insertion of the curved lateral osteotomes 300 into the knee. A second side of the elongated cutting portion 308a,b, opposite the edge 306a,b, is a flat portion 310a,b.

As illustrated in FIG. 4, the distal end 304a,b of the curved later osteotome 301a,b is capped with a flat triangular portion 312a,b that forms a point 314a,b at an end of the edge 306a,b. The flat portion 310a,b and the flat triangular portion 312a,b are flat to prevent the curved later osteotome 301a,b from penetrating through the knee and damaging other areas of the knee (e.g., ligaments, skin, etc.).

As illustrated in FIG. 5, the curved lateral osteotomes 300 can be used during the prosthetic implant revision. More specifically, the first curved lateral osteotome 301a can be used for a right side of the tibial implant 200 so that the edge 306a can cut a tray portion of the tibial implant from the bone/prosthetic while the flat portion 310a and the flat triangular portion 312a prevent the curved lateral osteotome 301a from causing tissue damage. The second curved lateral osteotome 301b can be used for a left side of the tibial implant 200 so that the edge 306b can cut a tray portion of the tibial implant from the bone/prosthetic while the flat portion 310b and the flat triangular portion 312b prevent the curved lateral osteotome 301a from causing tissue damage.

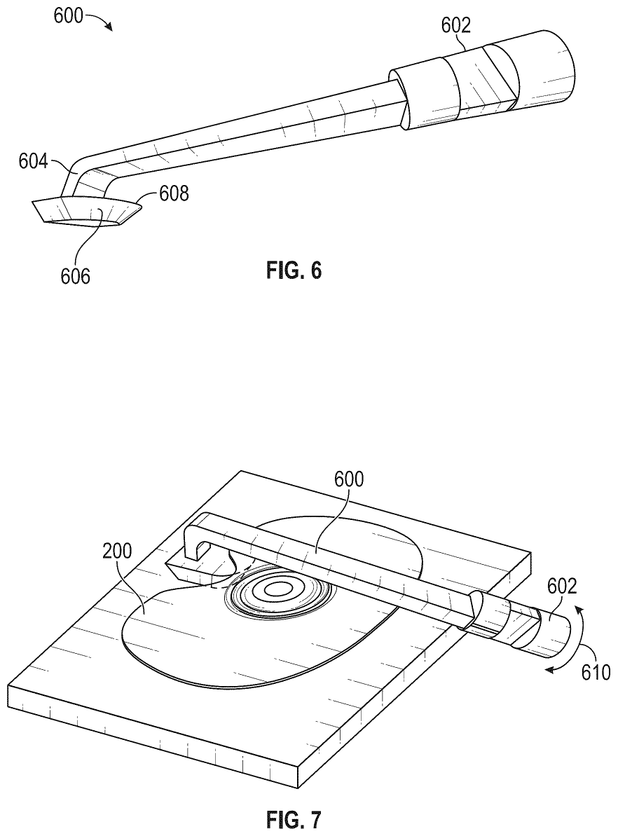

FIG. 6 illustrates a perspective view of a posterior osteotome 600 with a radial blade according to one or more aspects of the disclosed subject matter, and FIG. 7 illustrates the posterior osteotome 600 with the radial blade inserted under the tibial implant 200 according to one or more aspects of the disclosed subject matter. With reference to FIGS. 6 and 7, the posterior osteotome 600 includes a proximal end 602 and a distal end 604. The distal end 604 is bent and an end of the bent portion is connected to a radial blade 606. The radial blade 606 includes an edge 608 that is sharpened for cutting. The sharpened edge 608 cuts the bone growth along the bone/implant interface. The edge 608 is pointed toward the proximal end 602. The radial blade 606 can be inserted into a knee to assist with prosthetic implant revision. For example, as illustrated in FIG. 7, the radial blade 606 can cut the tibial implant from the bone on a posterior side of the knee. The posterior side of the knee is covered by tissue and important structure (e.g., the posterior cruciate ligament, blood vessels, arteries, nerves, etc.). The posterior osteotome 600 allows for cutting under the tibial tray on a posterior side of the knee while limiting exposure to the tissue and important structure. The radial blade 606 also allows efficient cutting in response to moving the proximal end 602 in a rotational direction as indicated by arrow 610.

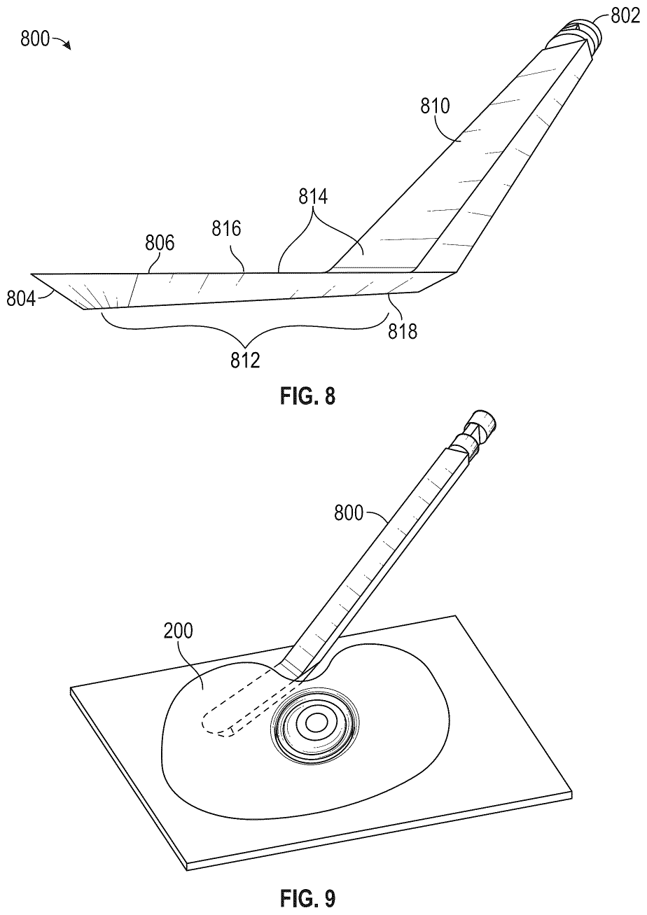

FIG. 8 illustrates a perspective view of an offset osteotome 800 according to one or more aspects of the disclosed subject matter, and FIG. 9 illustrates the offset osteotome 800 inserted under the tibial implant 200 according to one or more aspects of the disclosed subject matter. With reference to FIG. 8 and FIG. 9, the offset osteotome 800 includes proximal and distal ends (802 and 804), with distal end 804 having an edge 806 that is inserted into a knee for prosthetic implant revision. The edge 806 is sharpened for cutting. The sharpened edge 806 cuts the bone growth along the bone/implant interface and otherwise allows for the insertion of the offset osteotome 800 into the knee. For example, the edge 806 is a radial blade. The edge 806 extends a predetermined distance along a first side of the offset osteotome 800, around the distal end 804, and extends the predetermined distance along a second side of the offset osteotome 800. In other words, the edge 806 defines an elongated cutting portion 812 of the offset osteotome 800. The elongated cutting portion 812 has a flat top 816 that tapers to a flat bottom 814. Additionally, a height of the elongated cutting portion 812 is shorter than a height of the shaft 810. The sharpened edge 806 cuts the bone growth along the bone/implant interface and otherwise allows for the insertion of the straight osteotome 800 into the knee. Additionally, the elongated cutting portion 812 is offset at a predetermined offset angle 814 from a shaft 810 of the offset osteotome 800. For example, the offset angle 814 can range from 35 degrees to 45 degrees.

As illustrated in FIG. 9, the offset osteotome 800 can be inserted into the knee to cut bone growth along the bone/implant interface under the tibial implant 200. The offset angle 814 can allow easier access for cutting in certain areas of the knee. For example, the back of the knee has many veins, arteries, nerves, and the posterior ligament. The offset of the offset osteotome 800 allows for minimal exposure and less risk while releasing the implant from the bone because the offset angle 814 makes it possible to work around the posterior part of the tibia (e.g., as illustrated in FIG. 9). Additionally, similarly to the edge 106 of the elongated cutting portion 112 described herein, the edge 806 of elongated cutting portion 812 is ideally situated to be able to cut in many directions while inserted in the knee. For example, considering movement that is parallel to the flat bottom 818, for example, the edge 806 allows the elongated cutting portion 812 to cut in a forward-backward direction (e.g., corresponding to arrow 202 of FIG. 2), a left-right direction (e.g., corresponding to arrow 204 of FIG. 2), and/or a rotational direction (e.g., corresponding to arrow 206 of FIG. 2) while inserted in the knee. It should be appreciated that the offset osteotome 800 can also be used for femoral knee implant removal, and it is advantageous for the same reasons described herein for tibial implant removal.

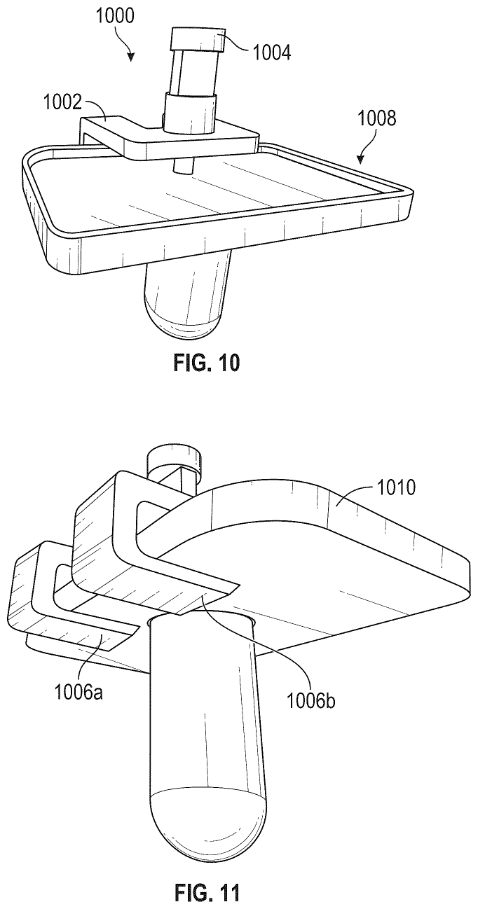

FIG. 10 illustrates a perspective view of a tibial extractor 1000 according to one or more aspects of the disclosed subject matter, and FIG. 11 illustrates a perspective view of the tibial extractor 1000 from below according to one or more aspects of the disclosed subject matter. With reference to FIG. 10 and FIG. 11, the tibial extractor 1000 includes a clamp portion 1002 and a connection portion 1004. The clamp portion 1002 includes feet 1006a and 1006b that hook under a front side relative to the knee of a tibial tray 1010 of a tibial implant 1008. The connection portion 1004 can be threaded through the clamp portion 1002. In one aspect, the connection portion 1004 can be threaded through the clamp portion 1002 and screwed into the tibial tray 1010. As a result, the connection portion 1004 allows pressure to be applied to the clamp portion 1002 such that the clamp portion 1002 is clamped to the tibial tray 1010. Then the tibial implant 1008 can be extracted by attaching a slap hammer or a handle, for example, to the connection portion 1004.

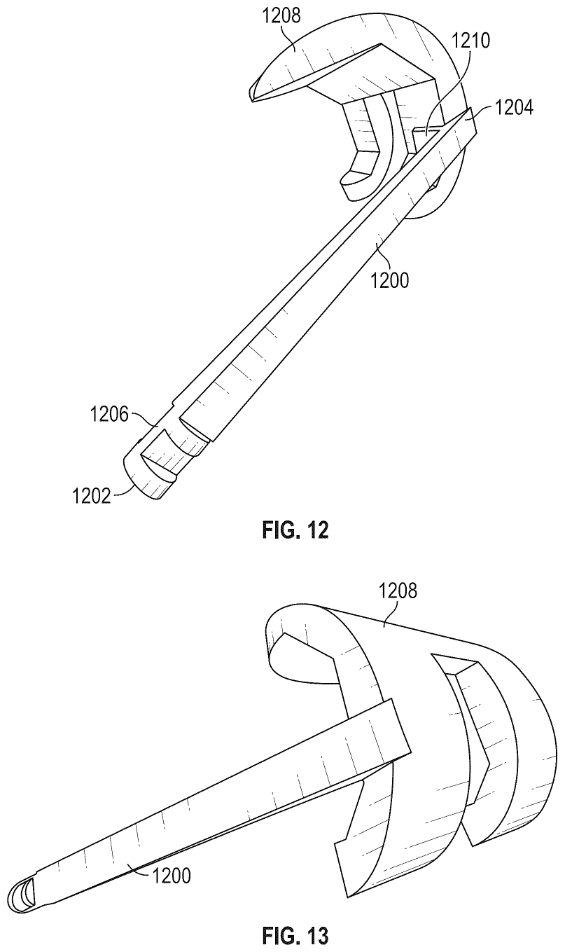

FIG. 12 illustrates a perspective view of an extractor 1200 according to one or more aspects of the disclosed subject matter, and FIG. 13 illustrates a perspective view of the extractor 1200 according to one or more aspects of the disclosed subject matter. Referring to FIG. 12 and FIG. 13, the extractor 1200 includes a proximal end 1202 and a distal end 1204. The proximal end 1202 includes a connection portion 1206. The distal end 1204 of the extractor 1200 includes an engagement portion 1210 that applies pressure to a femoral knee implant 1208 for removal. The engagement portion 1210 is a predetermined length so pressure can be applied closer to a center of the femoral knee implant 1208 rather than just an edge of the femoral knee implant 1208. For example, the length of the engagement portion 1210 can be 10-15 mm. This allows the femoral knee implant 1208 to be removed in line with bone and prevents the femoral implant 1208 from rocking on the bone. In one aspect, the extractor 1200 can also be used for a tibial implant.

Having now described embodiments of the disclosed subject matter, it should be apparent to those skilled in the art that the foregoing is merely illustrative and not limiting, having been presented by way of example only. Thus, although particular configurations have been discussed herein, other configurations can also be employed. Numerous modifications and other embodiments (e.g., combinations, rearrangements, etc.) are enabled by the present disclosure and are within the scope of one of ordinary skill in the art and are contemplated as falling within the scope of the disclosed subject matter and any equivalents thereto. Features of the disclosed embodiments can be combined, rearranged, omitted, etc., within the scope of the invention to produce additional embodiments. Furthermore, certain features may sometimes be used to advantage without a corresponding use of other features. Accordingly, Applicant(s) intend(s) to embrace all such alternatives, modifications, equivalents, and variations that are within the spirit and scope of the disclosed subject matter.

1. A tool for removal of a tibial implant, comprising:
a proximal end; and
a distal end having an elongated cutting portion, wherein the elongated cutting portion has an edge sharpened for cutting extending a predetermined distance on a first side of the tool, around the distal end, and extending the predetermined distance on a second side of the tool.

2. The tool of claim 1, wherein the edge is a radial blade.

3. The tool of claim 1, wherein the elongated cutting portion has a first flat surface on a first side of the tool, a second flat surface on a second side of the tool, wherein the second side is opposite of the first side.

4. The tool of claim 3, wherein the first flat surface tapers to the second flat surface.

5. The tool of claim 1, wherein, while the tool is inserted in a knee for removing the tibial implant, in response to movement in a forward-backward direction, the edge cuts in a corresponding direction.

6. The tool of claim 1, wherein, while the tool is inserted in a knee for removing the tibial implant, in response to movement in a left-right direction, the edge cuts in a corresponding direction.

7. The tool of claim 1, wherein, while the tool is inserted in a knee for removing the tibial implant, in response to movement in a rotational direction, the edge cuts in a corresponding direction.

8. The tool of claim 1, wherein the proximal end includes a connection interface.

9. The tool of claim 8, further comprising:
a shaft between the distal end and the connection interface.

10. The tool of claim 9, wherein a height of the elongated cutting portion is shorter than a height of the shaft.

11. A tool for removal of a tibial implant, comprising:
a proximal end;
a distal end having an elongated cutting portion, wherein the elongated cutting portion has an edge sharpened for cutting extending a predetermined distance on a first side of the tool, around the distal end, and extending the predetermined distance on a second side of the tool; and
a shaft between the proximal end and the distal end,
wherein the elongated cutting portion is offset by an offset angle relative to the shaft.

12. The tool of claim 11, wherein the offset angle ranges from 35 degrees to 45 degrees.

13. The tool of claim 11, wherein the edge is a radial blade.

14. The tool of claim 11, wherein the elongated cutting portion has a first flat surface on a first side of the tool, a second flat surface on a second side of the tool, wherein the second side is opposite of the first side.

15. The tool of claim 14, wherein the first flat surface tapers to the second flat surface.

16. The tool of claim 11, wherein, while the tool is inserted in a knee for removing the tibial implant, in response to movement in a forward-backward direction, the edge cuts in a corresponding direction.

17. The tool of claim 11, wherein, while the tool is inserted in a knee for removing the tibial implant, in response to movement in a left-right direction, the edge cuts in a corresponding direction.

18. The tool of claim 11, wherein, while the tool is inserted in a knee for removing the tibial implant, in response to movement in a rotational direction, the edge cuts in a corresponding direction.

19. The tool of claim 11, wherein the proximal end includes a connection interface.

20. The tool of claim 11, wherein a height of the elongated cutting portion is shorter than a height of the shaft.
